(19) United States
(12) Patent Application Publication
(10) Pub. No.: US 2022/0193288 A1
(43) Pub. Date: Jun. 23, 2022

(54) CART SANITIZER/WASHER

(71) Applicant: Techni LLC, Foster City, CA (US)

(72) Inventors: Nicholaos Dimitrious Fanourgiakis, Foster City, CA (US); John Dimitrious Fanourgiakis, Foster City, CA (US); Manoli Dimitrious Fanourgiakis, Foster City, CA (US); George Dimitrious Fanourgiakis, Foster City, CA (US)

(73) Assignee: .Techni LLC, Foster City, CA (US)

(21) Appl. No.: 17/561,339

(22) Filed: Dec. 23, 2021

Related U.S. Application Data
(60) Provisional application No. 63/130,398, filed on Dec. 23, 2020.

(51) Int. Cl. A61L 2/18; A61L 2/10; B60S 3/04

(52) U.S. Cl. CPC A61L 2/18; A61L 2/10; B60S 3/04; A61L 2202/17; A61L 2202/122; A61L 2202/15; A61L 2202/11

(57) ABSTRACT

A cart sanitizer/washer is disclosed. The cart sanitizer/washer includes a sanitization and wash chamber formed by a right sidewall panel, a left sidewall panel, a front sidewall panel, a rear sidewall panel, a ceiling panel, and a floor. Entry and exit ramps may be included to facilitate the rolling of one or more carts into the chamber. The sanitizer/washer includes a detergent container, a rinse agent container, a sanitizer container, a detergent pump, a rinse agent pump, a circulation pump, a sanitizer pump, and a drain pump in a compartment located above the chamber. An Ultraviolet (UV) Germicidal Irradiation (UVGI) system may be included within the chamber to provide an alternative meaning of sanitizing the cart positioned within the chamber with one or more lamp.

### CROSS-REFERENCE TO RELATED APPLICATION

This application claims the benefit of U.S. Provisional Patent Application Ser. No. 63/130,398, filed Dec. 23, 2020, which is incorporated by reference herein in its entirety.

### BACKGROUND

The present invention relates generally to shopping carts and, more particularly, a system and method for washing and sanitizing shopping carts.

Shopping carts are ubiquitous. They are used in commerce by merchants and shoppers for the placement and transport of merchandise that is to be stocked or purchased. They are commonly used in grocery stores (i.e., supermarkets), department stores, and various other types of business establishments.

It has been scientifically shown that shopping carts accumulate substantial concentrations of germs, viruses, and other disease agents that can spread infection amongst those who use the shopping carts by transference through touch. To address this issue, business establishments have provided a variety of attempted solutions to avert the spread of disease which include providing wipes and disinfectants for use, but these are largely overlooked and impractical. Customers would, instead, prefer to use a cart that they know is clean.

### SUMMARY

Embodiments of the inventive concepts disclosed herein are directed to a cart sanitizer/washer system. This system could be configured to perform wash/rinse and/or sanitizer operations to prevent the spread of germs, viruses, and other disease agents.

In one aspect, embodiments of the inventive concepts disclosed herein are directed to a system for sanitizing a cart. The system could include a sanitization chamber formed by a right sidewall panel, a left sidewall panel, a front sidewall panel, a rear sidewall panel, a ceiling panel, and a floor; a sanitizer container configured to store sanitizer for killing at least one of viruses, bacteria, and microorganisms on the cart; and a pump configured to receive sanitizer from the container and deliver the sanitizer to the sanitization chamber via a conduit. Also, the system may deliver sanitizer only in a sanitization cycle without a necessity of delivering detergent and rinse agents having wash and rinse cycles; in other words, the system may be employed to perform a sanitization cycle only.

In a further aspect, embodiments of the inventive concepts disclosed herein are directed to a second system for sanitizing a cart. The second system could include the chamber discussed in the preceding paragraph; and an Ultraviolet (UV) Germicidal Irradiation (UVGI) system.

In a further aspect, embodiments of the inventive concepts disclosed herein are directed to a portable system for washing and sanitizing a cart. The portable system could include the chamber of the preceding paragraphs; a compartment above the wash and sanitization chamber housing a wash detergent container and pump, a rinse agent container and pump, a sanitizer container and pump, a circulation pump, and a drain pump; a plurality of conduits configured to deliver wash detergent, rinse agent, and sanitizer to the wash and sanitization chamber; and a base configured for being lifted, transported, and positioned into a stationary position by a pallet jack. In an additional embodiment, the chamber could include the UVGI system described above.

The subject matter disclosed in U.S. Pat. Nos. 8,480,818 and 9,505,035 (singularly or collectively, “prior art”) are hereby incorporated by reference in their entireties.

### BRIEF DESCRIPTION OF THE DRAWINGS

For a fuller understanding of the inventive embodiments, reference is made to the following description taken in connection with the accompanying drawings in which:

FIGS. 1A through 1D illustrate exemplary cart sanitizer/washer systems, in accordance with some embodiments;

FIGS. 2A through 2C illustrate three perspective views a cart sanitizer/washer system, in accordance with some embodiments;

FIGS. 3A through 3C illustrate three perspective views a base employed by the cart sanitizer/washer system, in accordance with some embodiments;

FIGS. 4A through 4E illustrate one perspective view and four side views of cart sanitizer/washer system, in accordance with some embodiments;

FIGS. 5A through 5C illustrate three views of detergent and rinse agent pumps and associated conduits, in accordance with some embodiments;

FIGS. 5D through 5F illustrate a circulation and sanitizer pumps and associated conduits, in accordance with some embodiments;

FIGS. 5G through 5I illustrate the circulation pump and an associated conduit, in accordance with some embodiments;

FIGS. 5J through 5K illustrate the sanitizer pump and an associated conduit, in accordance with some embodiments;

FIGS. 5L through 5N illustrate a drain pump and an associated conduit, in accordance with some embodiments;

FIGS. 5O through 5Q illustrate the drain pump and an associated conduit; and

FIGS. 6A through 6E illustrate a distribution box and associated conduits, in accordance with some embodiments.

### DETAILED DESCRIPTION

In the following description, several specific details are presented to provide a thorough understanding of embodiments of the inventive concepts disclosed herein. One skilled in the relevant art will recognize, however, that embodiments of the inventive concepts disclosed herein can be practiced without one or more of the specific details, or in combination with other components, etc. In other instances, well-known implementations or operations are not shown or described in detail to avoid obscuring aspects of various embodiments of the inventive concepts disclosed herein. U.S. Pat. Nos. 8,480,818 and 9,505,035 (singularly or collectively, “prior art”) are incorporated by reference herein in their entirety.

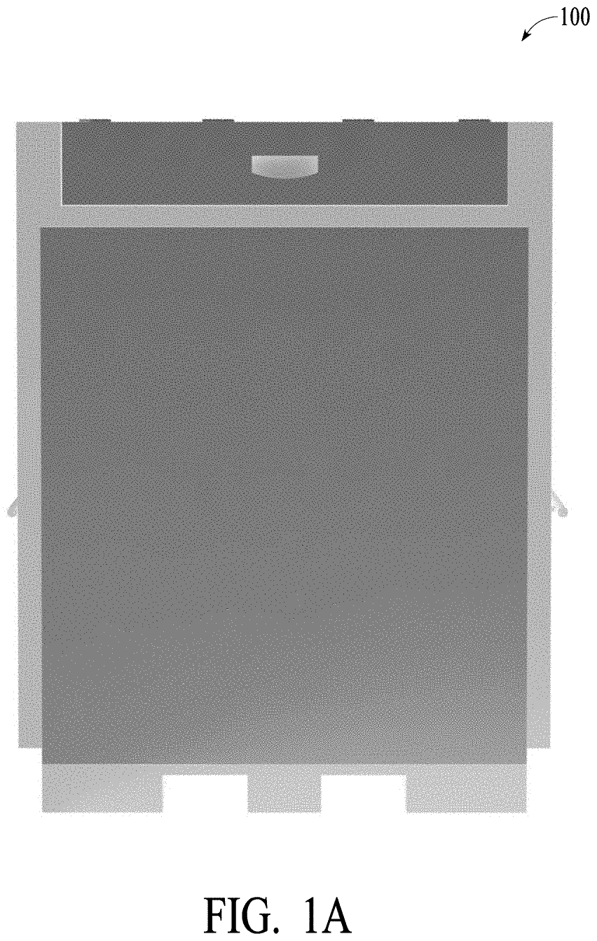

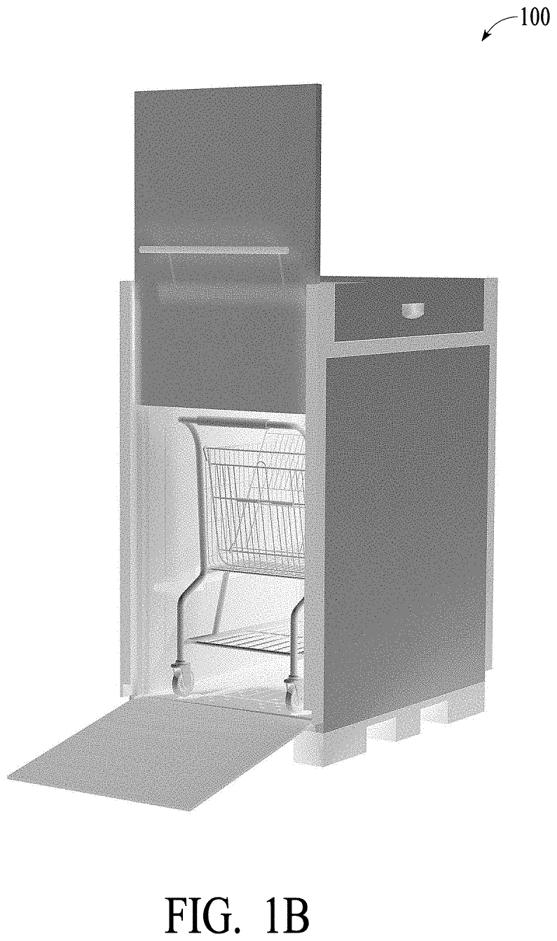

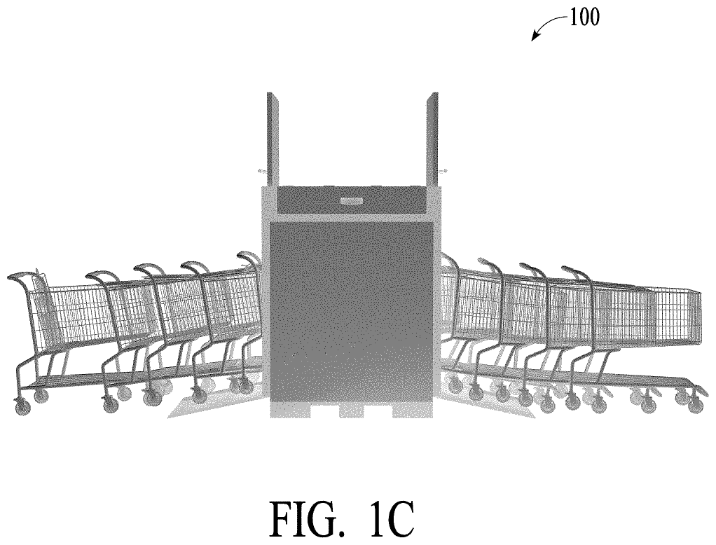

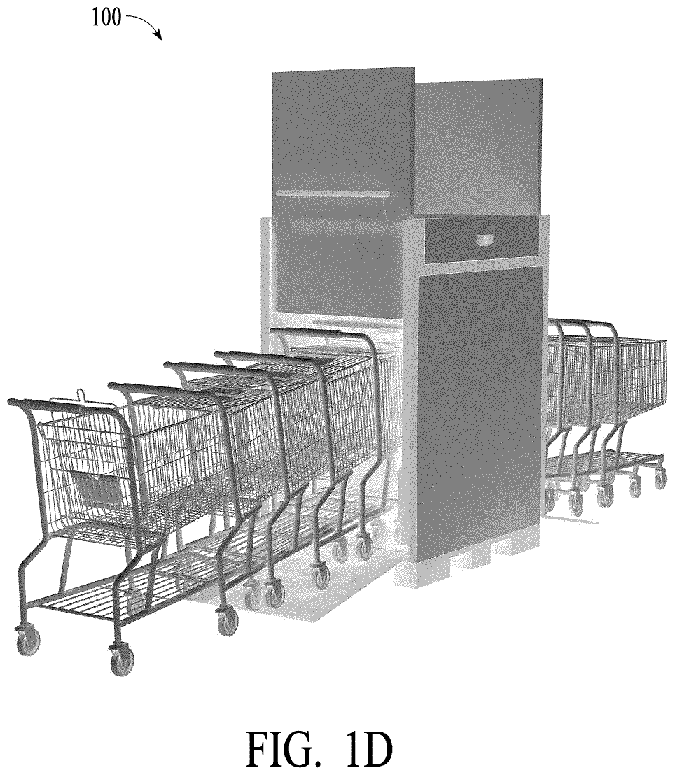

Referring now to FIGS. 1A-1D, a cart sanitizer/washer system 100 suitable for implementation of the inventive concepts described herein is illustrated. FIG. 1A illustrates a side view of the cart sanitizer/washer disclosed herein; FIG. 1B illustrates one employment of the cart sanitizer/washer with one door in which a single cart may be washed and/or sanitized FIGS. 1C-1D illustrate a second employment of the cart sanitizer/washer with front and rear doors in which horizontal stack of carts may sanitized by travelling through cart sanitizer/washer.

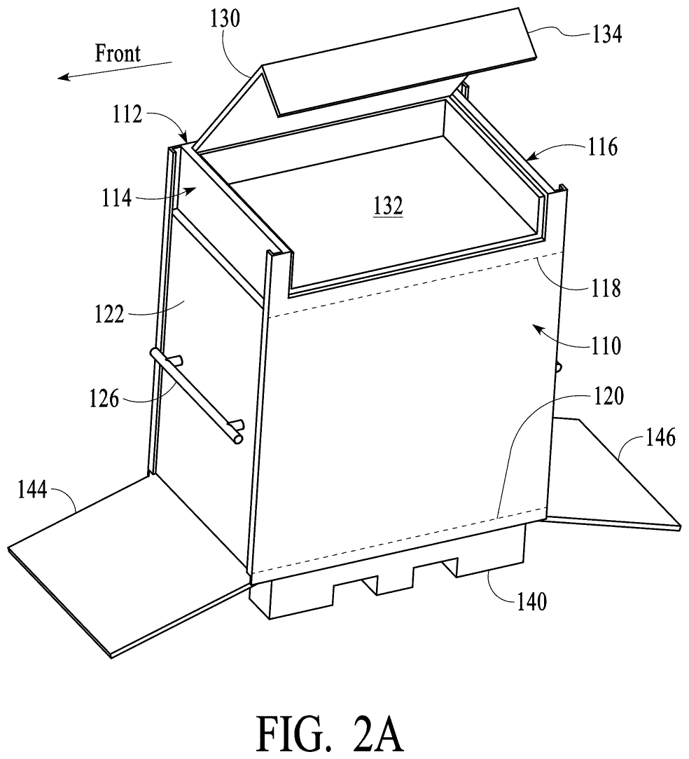

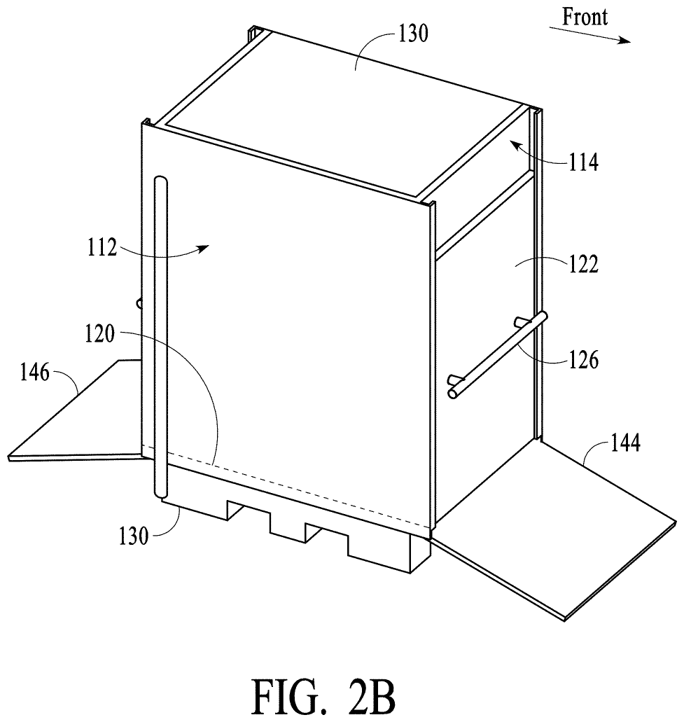

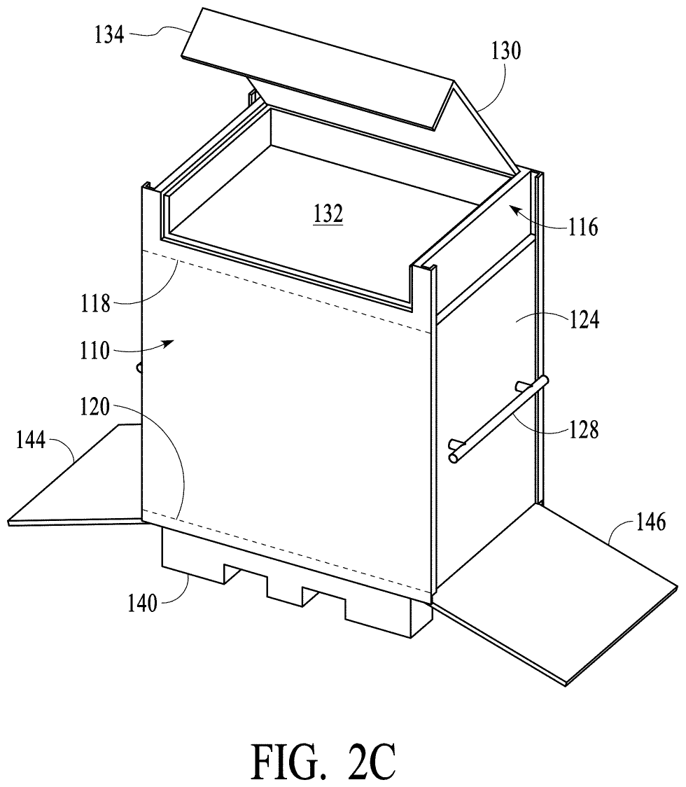

Referring now to FIGS. 2A-2C, three perspective views a cart sanitizer/washer system 100 are presented. Cart sanitizer/washer system 100 includes a sanitization/wash chamber formed by a right sidewall panel 110, a left sidewall panel 112, a front sidewall panel 114, a rear sidewall panel 116, a ceiling panel 118, and a floor 120. A front door 122 and a rear door 124 provide an entrance and exit, respectively, into and from the sanitization/wash chamber. Front door 122 and rear door 124 are each moveable vertically within tracks disposed within right sidewall panel 110 and left sidewall panel 112 (not shown) by a user lifting it vertically by its respective handle 126 and 128, respectively, until it is disengaged.

Cart sanitizer/washer system 100 includes a housing above ceiling panel 118 of the sanitization/wash chamber, where the housing includes an access cover 130 and platform 132. Left sidewall panel 112, front sidewall panel 114, and/or rear sidewall panel 116 have been modified to pivotally facilitate the raising and lowering of access cover 130. As shown, right sidewall panel 110 has been configured (or shaped) to receive a downward member 134 to provide a user more space to access components located within the housing while maintaining the ability to completely enclose the housing when access cover 130 has been lowered into its closed position.

It should be noted that, although the illustrated access cover 130 pivotally moves towards left sidewall panel 112 when raised, the embodiments herein are not limited to this configuration. In some configurations, right sidewall panel 110, left sidewall panel 112, and/or rear sidewall panel 116 may be modified to pivotally facilitate the raising and lowering of access cover 130, and front sidewall panel 114 may be shaped to receive a downward member 134.

In some configurations, left sidewall panel 112, front sidewall panel 114, and/or rear sidewall panel 116 may be modified to pivotally facilitate the raising and lowering of access cover 130, and right sidewall panel 112 may be shaped to receive a downward member 134. In some configurations, right sidewall panel 110, left sidewall panel 112, and/or front sidewall panel 114, may be modified to pivotally facilitate the raising and lowering of access cover 130, and rear sidewall panel 116 may be shaped to receive a downward member 134.

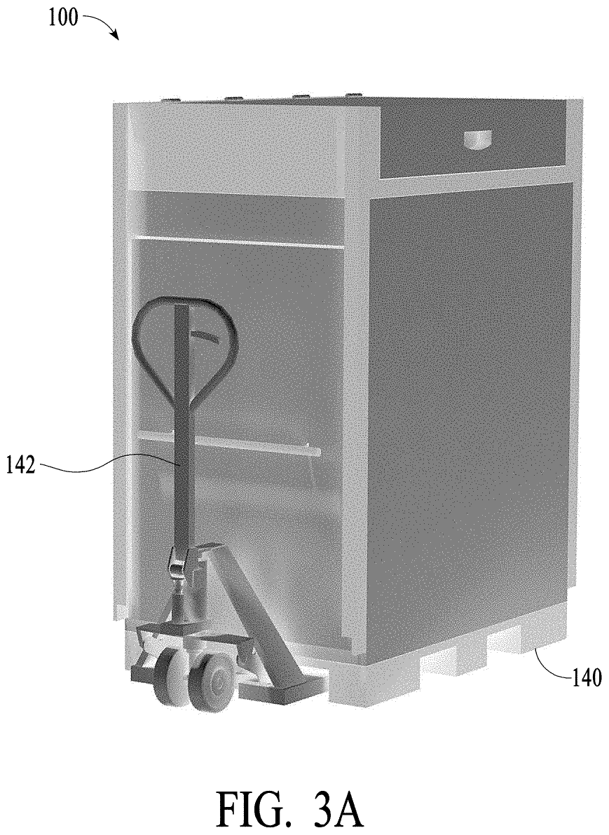

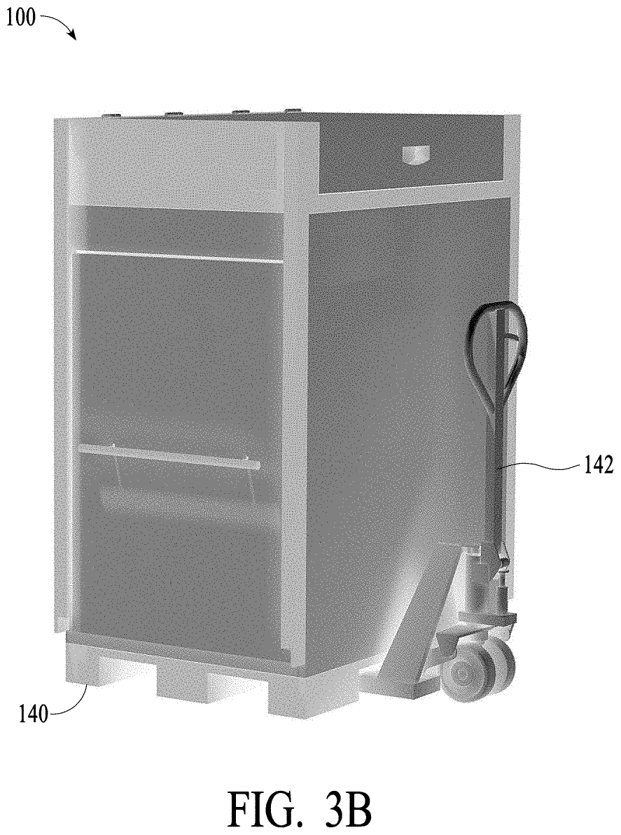

Cart sanitizer/washer system 100 includes a base 140 which could facilitate the portability of the system. Referring now to FIGS. 3A-3C, the underside of base 140 has been configured to receive forks of a standard pallet jack 142. Because the sanitization/wash chamber has elevated cart sanitizer/washer system 100 above the surface, ramps 144 and 146 may be employed to facilitate a cart's rolling entrance into and exit from the sanitization/wash chamber.

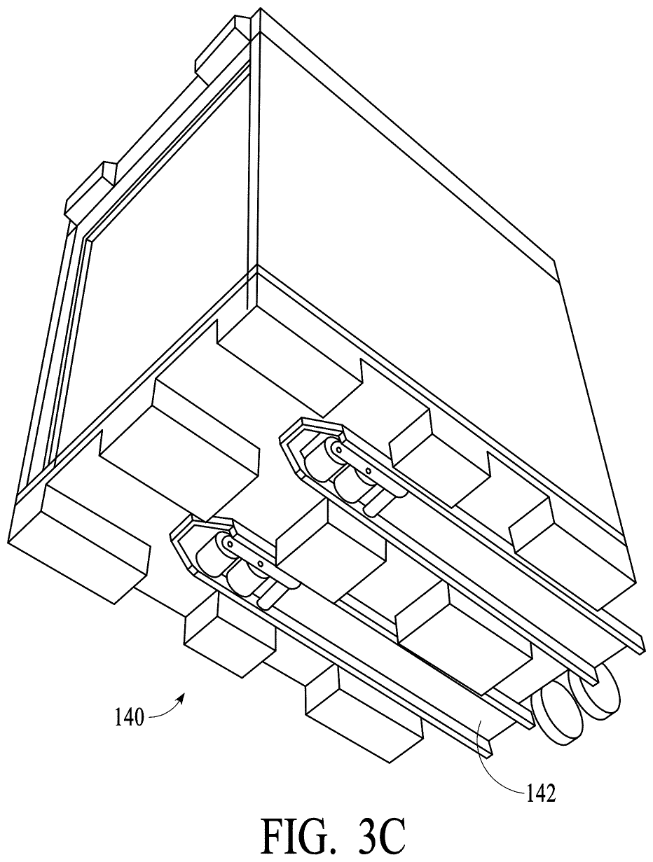

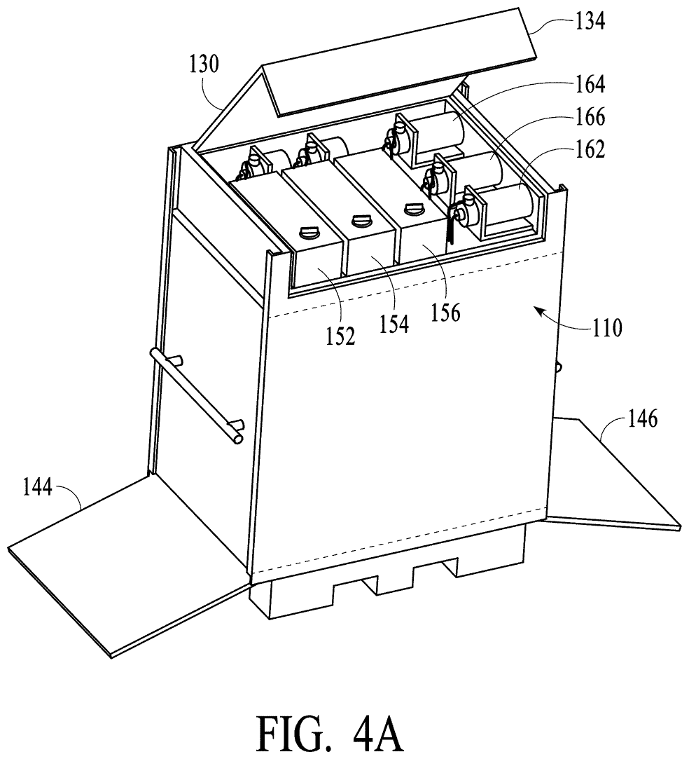

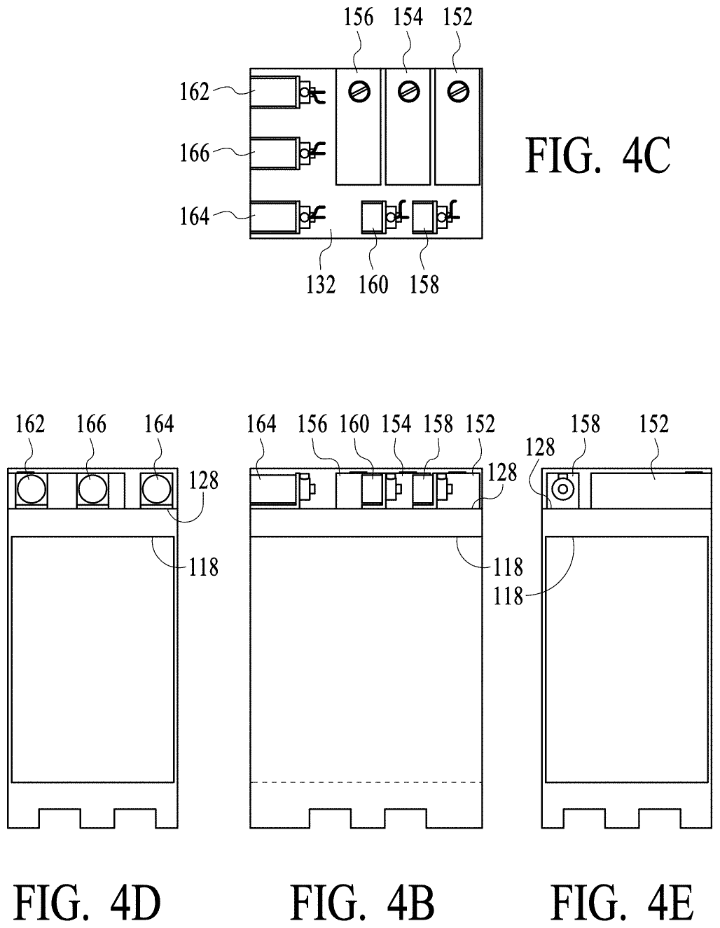

Referring now to FIGS. 4A-4E, one perspective view and four side views of cart sanitizer/washer system 100 are illustrated. As shown in FIG. 4A, an access cover 130 has been placed into its raised position to allow a user access to a plurality of components found on platform 132 including a detergent container 152 for receiving/storing washing detergent; a rinse agent container 154 for receiving/storing rinsing agent; a sanitizer container 156 for receiving/storing sanitizer for killing viruses, bacteria, and/or microorganisms on hands; a detergent pump 158 for delivering the detergent to the sanitization/wash chamber from detergent container 152; a rinse agent pump 160 for delivering the rinse agent to the sanitization/wash chamber from rinse agent container 154; a circulation pump 162 for circulating both a water-detergent mixture in a wash cycle and a water-rinse agent mixture in a rinse cycle; a sanitizer pump 164 for delivering the sanitizer from sanitizer container 156 in a sanitization cycle; and a drain pump 166 for draining the water-detergent and water-rinse agent mixtures from the sanitization/wash chamber upon the completion of the wash and rinse cycles, respectively, are completed. It should be noted the general or generic illustrations of pumps having input and outlet ports are presented for the purpose of illustration only and not representative of any specific pump.

It should also be noted that, although the discussion herein is drawn to these components being housing located above the sanitization/wash chamber, the embodiments herein are not limited to this configuration. For instance, these could be located in a housing this is adjacent to a side of right sidewall panel 110 or left sidewall panel 112. In such configuration, however, the routing of one or more conduits may have to be modified from those discussed below to accommodate this configuration.

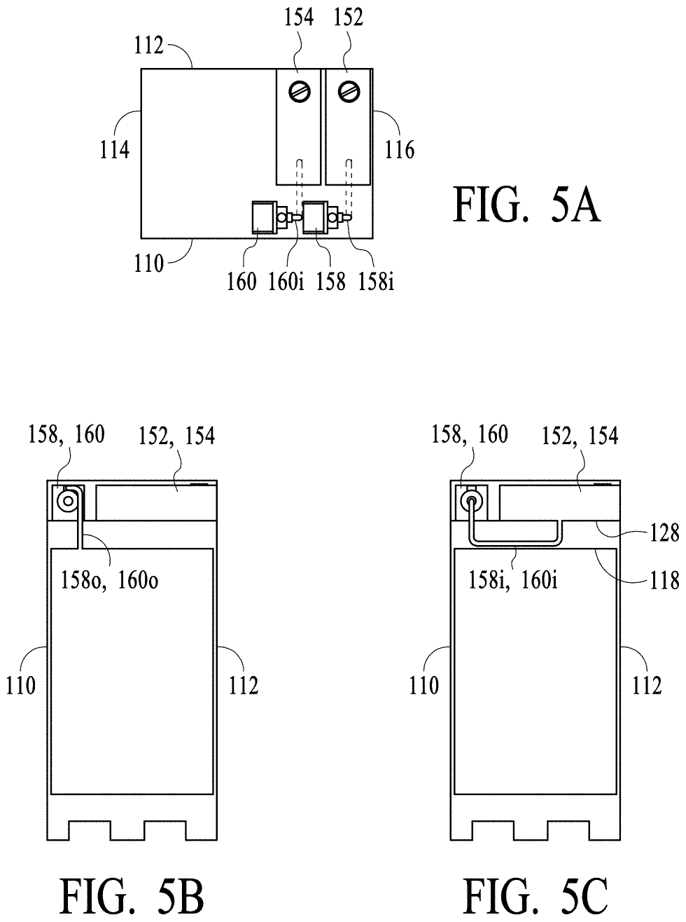

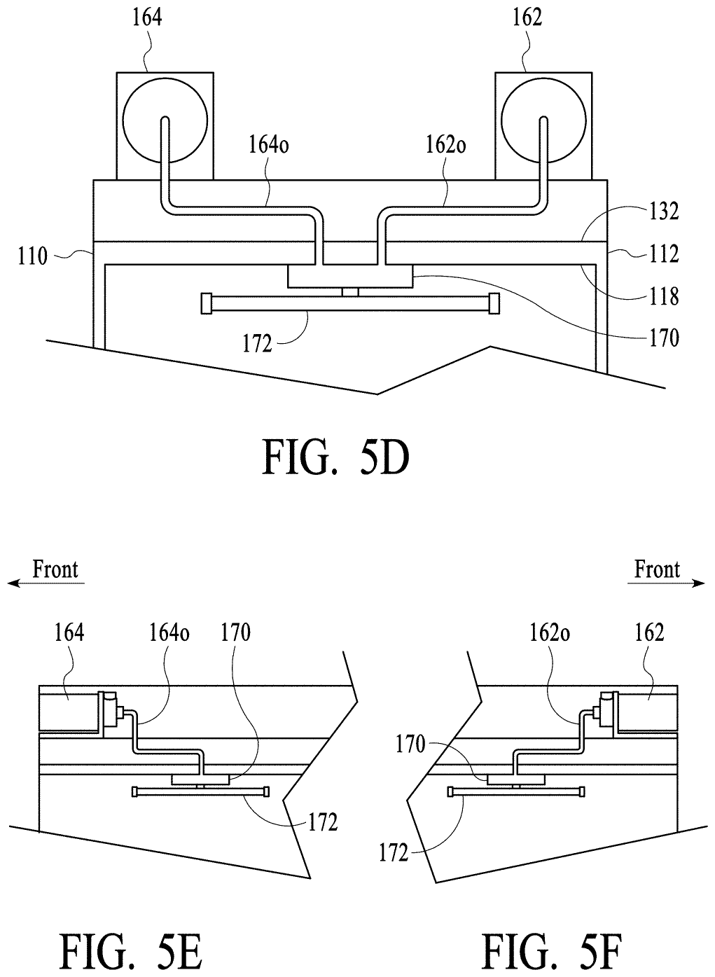

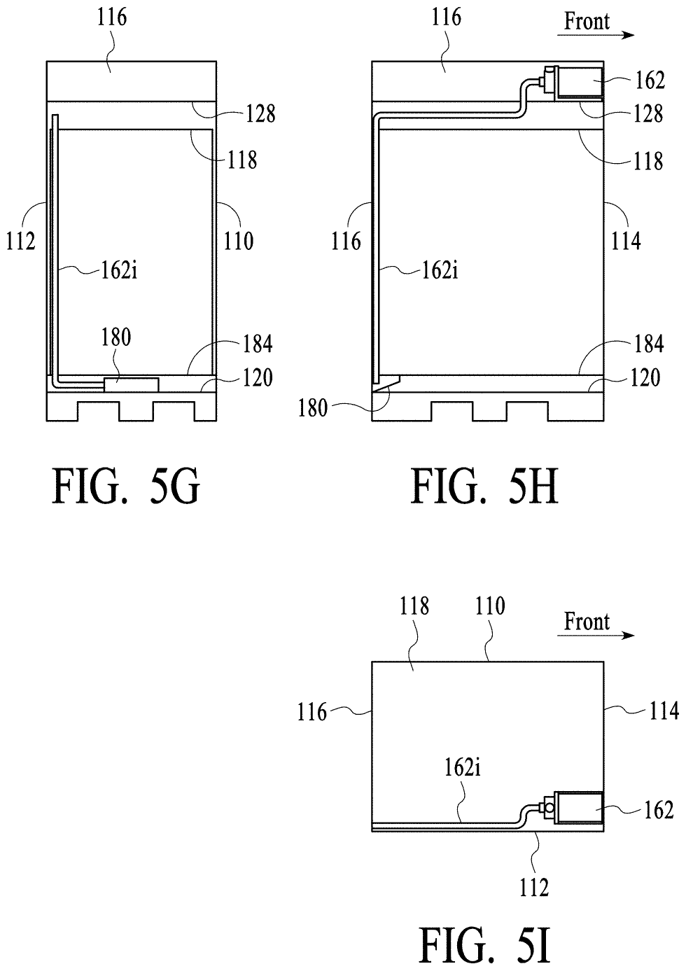

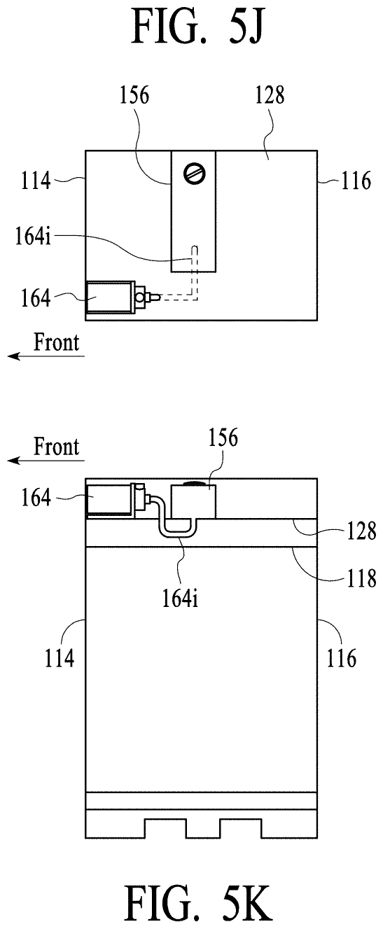

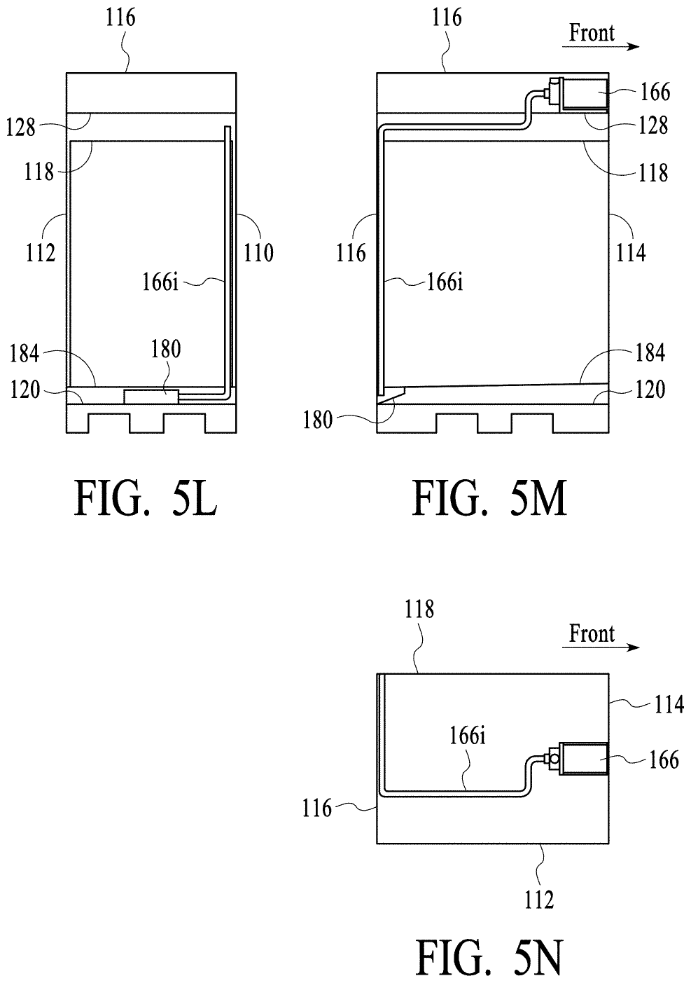

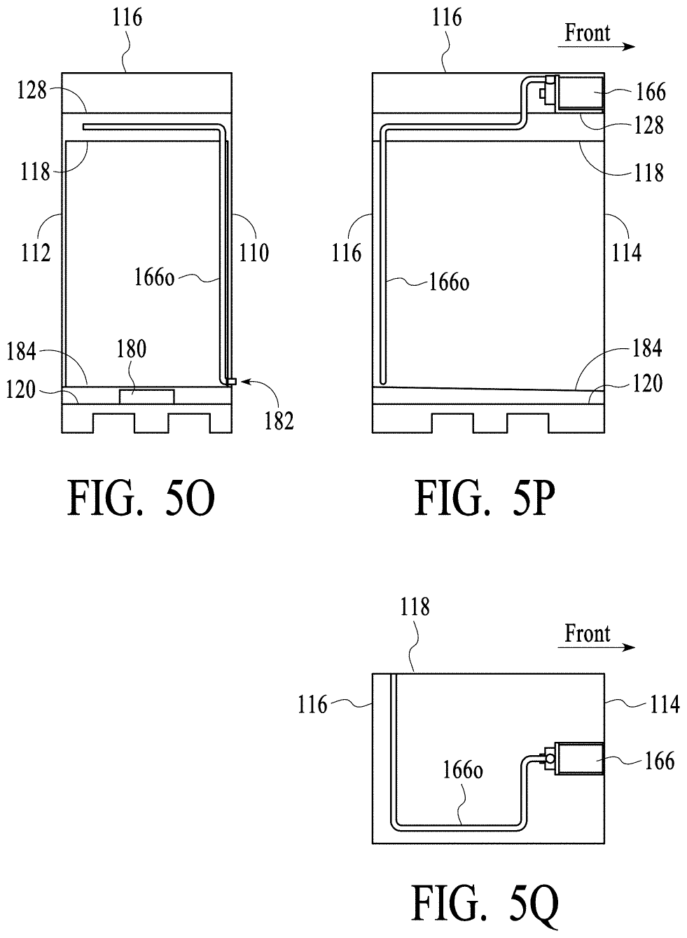

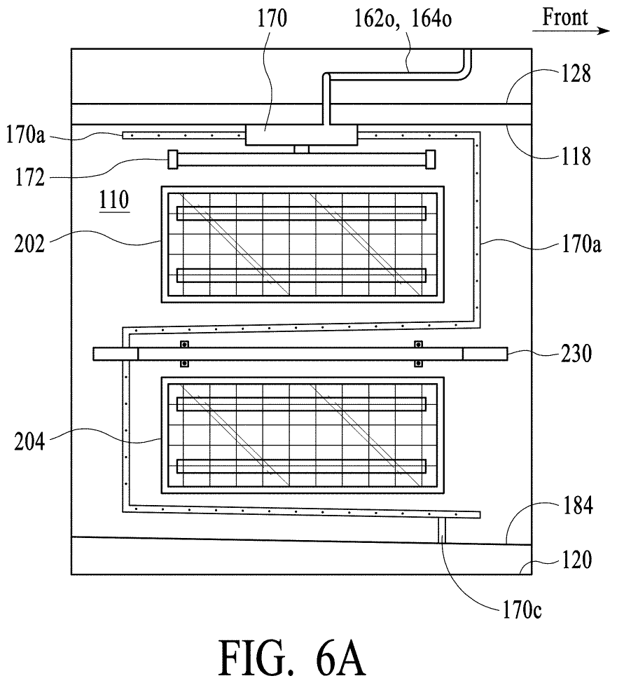

Referring now to FIGS. 5A-5C (40-4Q), detergent pump 158 and its conduits 158i and 158o are illustrated, and rinse agent pump 160 and its conduits 160i and 160o are illustrated. As shown, detergent pump 158 receives detergent from detergent container 152 via conduit 158i traversing downward through an opening in platform 132 from an access port of detergent container 152, then towards detergent pump 158 within the space between ceiling panel 118 and platform 132, and then upward to the input port of detergent pump 158 through an opening in platform 132. In its delivery operation, detergent pump 158 delivers the detergent into the sanitization/wash chamber through delivery port 158a of ceiling panel 118 via conduit 158o traversing downward from detergent pump 158 through an opening in platform 132, where it is mixed with water received by the cart sanitizer/washer system 100 through an inlet port (not shown). In some embodiments, simultaneous receipt and delivery operations of detergent pump 158 (and delivery of water into the sanitization/wash chamber) may be controlled manually and/or automated through the use of software to operate detergent pump 158 at any time during a wash cycle including the beginning.

Similarly, rinse agent pump 160 receives rinse agent from rinse agent container 154 via conduit 160i traversing downward through an opening in platform 132 from an access port of rinse agent container 154, then towards rinse agent pump 160 within the space between ceiling panel 118 and platform 132, and then upward to the input port of rinse agent pump 160 through an opening in platform 132. In its delivery operation, rinse agent pump 160 delivers the rinse agent into the sanitization/wash chamber through delivery port 160a of ceiling panel 118 via conduit 160o traversing downward from rinse agent pump 160 through an opening in platform 132. In some embodiments, simultaneous receipt and delivery operations of rinse agent pump 160 may be controlled manually and/or automated through the use of software to operate rinse agent pump 158 at any time during a rinse cycle including the beginning.

Referring now to FIGS. 5D-5F (4A-4C), circulation pump 162 and one of its conduits 162o are illustrated, and sanitizer pump 164 and one of its conduits 1640 are illustrated. As shown, circulation pump 162 delivers the water-detergent mixture (during the wash cycle) and the water-rinse agent mixture (during the rinse cycle) to a distribution box 170 via conduit 162o traversing downward through an opening in platform 132 from an output port of circulation pump 162, then towards distribution box 170 within the space between ceiling panel 118 and platform 132, and then downward through an opening in ceiling panel 118 until reaching distribution box 170 adapted to engage conduit 162o and receive the water-detergent mixture and the water-rinse agent. In some embodiments, these delivery operations of circulation pump 162 may be controlled manually and/or automated through the use of software to operate circulation pump 162 during the respective wash and rinse cycles. It should be noted only water may be circulated during the wash and rinse cycles for a short period of time in which the detergent and rinse agent, respectively, have not yet been delivered into the sanitization/wash chamber. For the purpose of illustration and not of limitation, distribution box 170 is coupled to ceiling panel 118 within the wash camber and rotating bar 172 extends from distribution box 170.

Sanitizer pump 164 delivers the sanitizer to distribution box 170 during a sanitation cycle via conduit 164o traversing downward through an opening in platform 132 from an output port of sanitizer pump 164, then towards distribution box 170 within the space between ceiling panel 118 and platform 132, and then downward through an opening in ceiling panel 118 until reaching distribution box 170 adapted to engage conduit 164o and receive the sanitizer. In some embodiments, this delivery operation of sanitizer pump 164 may be controlled manually and/or automated through the use of software to operate sanitizer pump 164 during the sanitization cycle.

Referring now to FIGS. 5G-5I (4D-4F), circulation pump 162 and one of its conduits 162i are illustrated. As illustrated, circulation pump 162 may receive the water-detergent mixture (during the wash cycle), the water-rinse agent mixture (during the rinse cycle), or the sanitizer (during the sanitization cycle) from a sump 180 via conduit 162i traversing along floor 120 until reaching left sidewall panel 112, then upward along left sidewall panel 112 and into the space between ceiling panel 118 and platform 132 through ceiling panel 118, then towards circulation pump 162 within the space, and then upward to an input port of circulation pump 162 through an opening in platform 132. In some embodiments, these receipt operations of circulation pump 162 is performed simultaneously with its delivery operation and controlled manually and/or automated through the use of software to operate circulation pump 162 during the respective wash, rinse, and sanitization cycles. It should be noted only water may be circulated during the wash and rinse cycles for a short period of time in which the detergent and rinse agent, respectively, have not yet been delivered into the sanitization/wash chamber.

Referring now to FIGS. 5J-5K (4M-4N), sanitizer pump 164 and one of its conduits 164i are illustrated. As illustrated, sanitizer pump 164 receives the sanitizer from sanitizer container 156 via conduit 164i traversing downward through an opening in platform 132 from an access port of sanitizer container 152, then towards sanitizer pump 164 within the space between ceiling panel 118 and platform 132, and then upward to the input port of sanitizer pump 164 through an opening in platform 132. In some embodiments, the receipt operation of sanitizer pump 164 is performed simultaneously with its delivery operation and controlled manually and/or automated through the use of software to operate sanitizer pump 164 during the sanitization cycle.

Referring now to FIGS. 5L-5N (4G-4I), drain pump 166 and one of its conduits 166i are illustrated. As illustrated, drain pump 166 may receive the water-detergent mixture (at the end of the wash cycle), the water-rinse agent mixture (at the end of the rinse cycle), and the sanitizer (at the end of the sanitization cycle) from sump 180 via conduit 166i traversing along floor 120 until reaching right sidewall panel 110, then upward along right sidewall panel 110 and into the space between ceiling panel 118 and platform 132 through an opening in ceiling panel 118, then within the space towards drain pump 166, and then upward to an input port of drain pump 166 through an opening in platform 132. In some embodiments, these receipt operations of drain pump 166 may be controlled manually and/or automated through the use of software to operate drain pump 166 at the end of the respective wash, rinse, and sanitization cycles.

Referring now to FIGS. 5O-5Q (4J-4L), drain pump 166 and one of its conduits 166o are illustrated. As illustrated, after drain pump 166 receives the water-detergent mixture, the water-rinse agent mixture, and the sanitizer during their respective cycles from sump 180, drain pump 166 delivers the mixtures and sanitizer to outlet 182 via conduit 166o traversing downward though an opening in platform 132 from an output port of circulation pump 162, then towards rear sidewall panel 116 within the space between ceiling panel 118 and platform 132, and then downward through ceiling panel 118 until reaching port 182 through which the water-detergent mixture, the water-rinse agent mixture, and the sanitizer is discharged from the sanitization/wash chamber and cart sanitizer/washer system 100. In some embodiments, these delivery operations of drain pump 166 is performed simultaneously with its receipt operation and controlled manually and/or automated through the use of software to operate drain pump 166 at the end of the respective wash, rinse, and sanitization cycles.

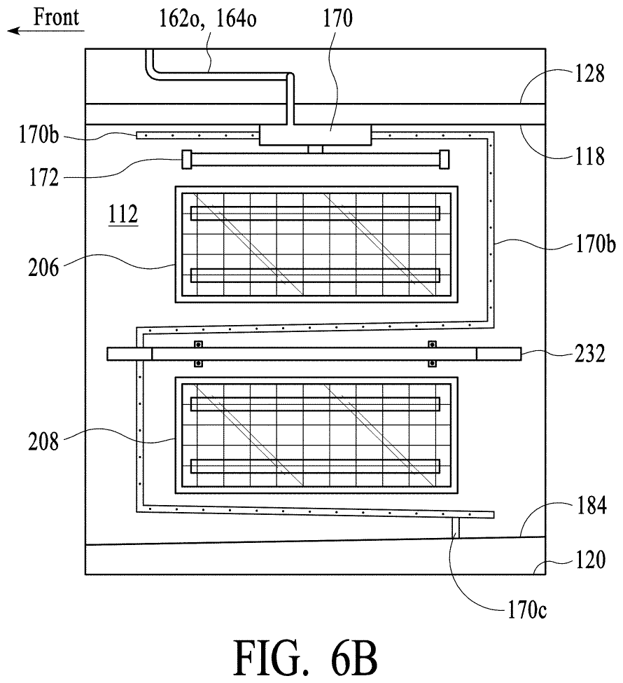

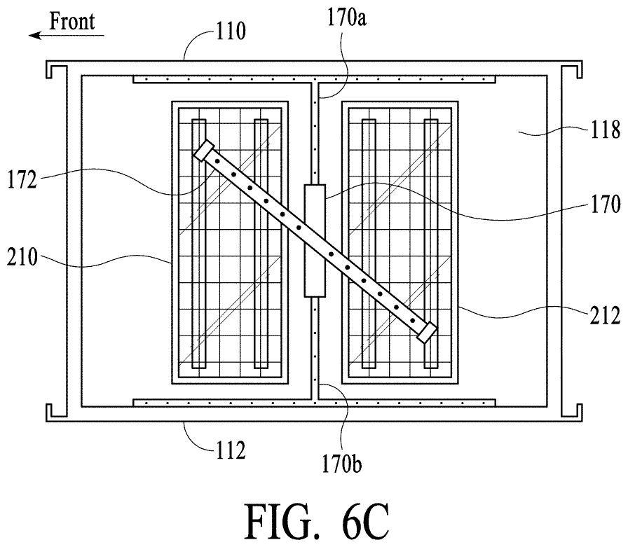

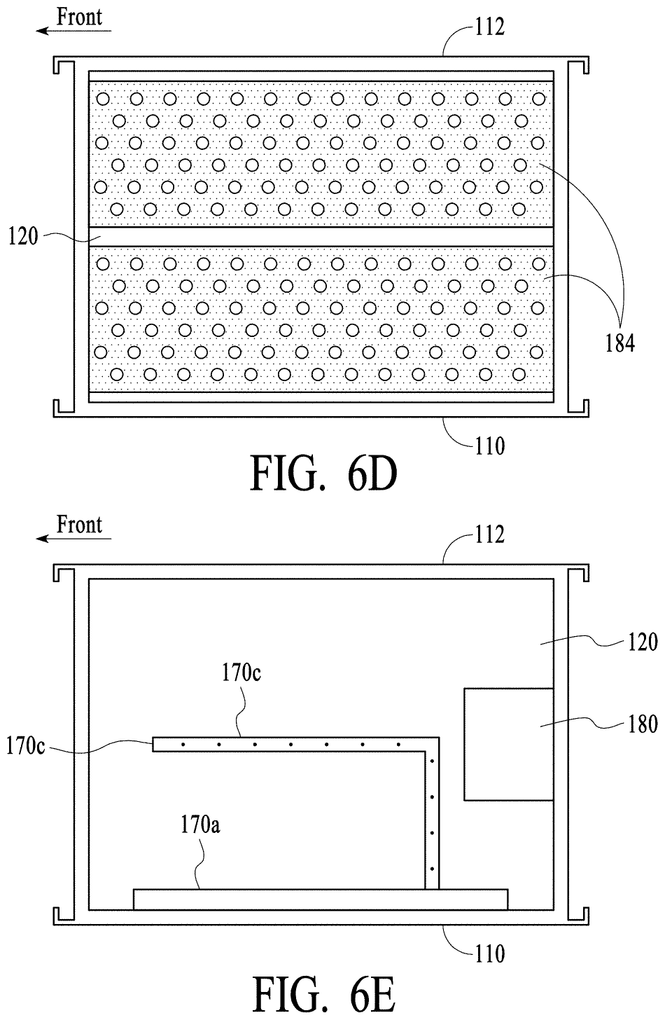

Referring now to FIGS. 6A-6E (5A-5E), distribution box 170, its conduits 170a and 170b, and rotating bar 172 are illustrated. As shown, distribution box 170 is coupled to ceiling panel 118 within the wash camber, and conduits 170a and 170b extend therefrom towards right sidewall panel 110 and left sidewall panel 112, respectively. When distribution box 170 receives the water-detergent mixture and the water-rinse agent mixture during wash and rinse cycles, respectively, from circulation pump 162, these mixtures are delivered to both right sidewall panel 110 and left sidewall panel 112 via conduits 170a and 170b, respectively; these mixtures are also delivered to a rotating bar 172 coupled to distribution box 170 configured to receive the mixtures and rotate or spin as a result of the force being imparted to the mixtures by circulation pump 162. Through a plurality of nozzles (represented by dots) found on conduits 170a and 170b and rotating bar 172, a cart(s) positioned inside of the sanitization/wash chamber in between guard rails 230 and 232 is sprayed with the water-detergent mixture during the wash cycle and the water-rinse agent mixture during the rinse cycle.

Similarly, when distribution box 170 receives the sanitizer from sanitizer pump 164 during the sanitization cycle, it is delivered to both right sidewall panel 110 and left sidewall panel 112 via conduits 170a and 170b, respectively; the sanitizer is also delivered to a rotating bar 172 configured to receive the sanitizer and rotate or spin as a result of the force being imparted to the sanitizer by sanitizer pump 164. Through a plurality of nozzles as shown, a cart(s) positioned inside of the sanitization/wash chamber is sprayed with sanitizer during the sanitization cycle.

In some embodiments, conduit 170a could include an extension 170c which traverses along floor 120 from right sidewall panel 110 to the middle on floor, and then towards the front. As observed, extension 170c could be located between pair of platforms 184 that are positioned above floor 120 and onto which the cart(s) are positioned; as shown, pair of platforms 184 include numerous openings to allow water-detergent and water-rinse mixtures and sanitizers that have sprayed the cart(s) to drip onto floor 120 and into sump 180; in some embodiments, floor 120 may be configured to slant rearward to aid in a gravitation flow of the water-detergent and water-rinse mixtures and sanitizers. Through a plurality of nozzles as shown, a cart(s) positioned inside of the sanitization/wash chamber may be sprayed from underneath with water-detergent mixture during the wash cycle, water-rinse agent mixture during the rinse cycle, and sanitizer during the sanitization cycle.

In an additional embodiment, sanitization may be provided by an Ultraviolet (UV) Germicidal Irradiation (UVGI) system employing one or more UVGI lights within the sanitization/wash chamber. As shown, six UVGI lights 202, 204, 206, 208, 210, 212. Each may be disposed and/or mounted to right sidewall panel 110, left sidewall panel 112, and/or ceiling panel 118, and oriented towards a cart(s) so that it is subjected to the UVGI emissions. In some embodiments, UVGI system and its emission of radiation from one or more of UVGI lights 202, 204, 206, 208, 210, 212 may be controlled manually and/or automated through the use of software during the sanitization cycle of the UVGI system.

As used herein, the term “embodiment” means an embodiment that serves to illustrate by way of example but not limitation.

It will be appreciated to those skilled in the art that the preceding examples and embodiments are exemplary and not limiting to the scope of the inventive concepts disclosed herein. It is intended that all modifications, permutations, enhancements, equivalents, and improvements thereto that are apparent to those skilled in the art upon a reading of the specification and a study of the drawings are included within the true spirit and scope of the inventive concepts disclosed herein. It is therefore intended that the following appended claims include all such modifications, permutations, enhancements, equivalents, and improvements falling within the true spirit and scope of the inventive concepts disclosed herein.

What is claimed is:

1. A system for sanitizing a cart, comprising:
a sanitization chamber formed by a right sidewall panel, a left sidewall panel, a front sidewall panel, a rear sidewall panel, a ceiling panel, and a floor;
a sanitizer container configured to store sanitizer for killing at least one of viruses, bacteria, and microorganisms on the cart; and
a pump configured to receive sanitizer from the container and deliver the sanitizer to the sanitization chamber via a conduit.

2. The system of claim 1, further comprising:
entrance and exit doors through which a cart enters and exits the sanitization chamber.

3. The system of claim 1, further comprising:
a supply of water, and
a detergent container configured to store wash detergent for cleaning the cart during a wash cycle; and
a circulation pump configured to receive the water and the detergent from the supply of water and the detergent container, respectively, and deliver a water-detergent mixture to a distribution box via a water-detergent conduit, whereby
the water-detergent mixture is distributed through distribution conduits configured with nozzles, thereby
spraying a cart positioned within the sanitization chamber with the water-detergent mixture.

4. A system for sanitizing a cart, comprising:
a sanitization chamber formed by a right sidewall panel, a left sidewall panel, a front sidewall panel, a rear sidewall panel, a ceiling panel, and a floor; and
an Ultraviolet (UV) Germicidal Irradiation (UVGI) system.

5. The system of claim 4, wherein the UVGI system is comprised of at least one UV lamp.

6. The system of claim 4, further comprising:
entrance and exit doors through which a cart enters and exits the sanitization chamber.

7. The system of claim 4, further comprising:
a supply of water, and
a detergent container configured to store wash detergent for cleaning the cart during a wash cycle; and
a circulation pump configured to receive the water and the detergent from the supply of water and the detergent container, respectively, and deliver a water-detergent mixture to a distribution box via a water-detergent conduit, whereby
the water-detergent mixture is distributed through distribution conduits configured with nozzles, thereby
spraying a cart positioned within the sanitization chamber with the water-detergent mixture.

8. A portable system for washing and sanitizing a cart, comprising:
a wash and sanitization chamber formed by a right sidewall panel, a left sidewall panel, a front sidewall panel, a rear sidewall panel, a ceiling panel, and a floor;
a compartment above the wash and sanitization chamber housing a wash detergent container and pump, a rinse agent container and pump, a sanitizer container and pump, a circulation pump, and a dump pump;
a plurality of conduits configured to deliver wash detergent, rinse agent, and sanitizer to the wash and sanitization chamber; and
a base configured for being lifted, transported, and positioned into a stationary position by a pallet jack.

9. The portable system of claim 8, further comprising:
a UVGI system for sanitizing a cart positioned within the wash and sanitization chamber

10. The system of claim 8, further comprising:
entrance and exit doors through which a cart enters and exits the sanitization chamber.
